(19) United States
(12) Patent Application Publication
(10) Pub. No.: US 2008/0147124 A1
(43) Pub. Date: Jun. 19, 2008

(54) BONE PLATE SYSTEM WITH SLIDABLE COMPRESSION HOLES

(76) Inventors: George J. HAIDUKEWYCH, Tampa, FL (US); Marc E. Ruhling, Goshen, IN (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/928,253

(22) Filed: Oct. 30, 2007

Related U.S. Application Data
(60) Provisional application No. 60/863,626, filed on Oct. 31, 2006.

(51) Int. Cl. A61B 17/58; A61B 17/56

(52) U.S. Cl. 606/280; 606/301

(57) ABSTRACT

A bone plate system includes a bone plate with movable portions and locking screw assemblies. The locking screw assemblies may be locked to the movable portions of the bone plate and then dynamized to either distract or compress bone fragments. The movable portions of the bone plate may be moved with respect to fixed portions of the bone plate through threaded spindle drive mechanisms or may be slidable on rails both before and after implantation. The locking screw assemblies include collets that may be pre-assembled with the bone screws. The collets are assemblies of inner and outer compressible members. The outer members have tabs to be received in grooves in bone screw holes of the bone plate. The inner members are sized and shaped to frictionally engage the bone screw when radially compressed.

### CROSS-REFERENCE TO RELATED APPLICATIONS

This application claims priority to U.S. Prov. App. No. 60/863,626 filed Oct. 31, 2006, entitled “BONE PLATE SYSTEM WITH SLIDABLE COMPRESSION HOLES,” which is incorporated by reference herein in its entirety.

### BACKGROUND OF THE INVENTION

The present invention relates to orthopaedic devices, and more particularly, to a bone plate system that allows for dynamizing bone screw positions for applying compression to a bone fracture site.

The skeletal system includes many long bones, which extend from the human torso. These long bones include the femur, fibula, tibia, humerus, radius and ulna. These long bones are particularly exposed to trauma from accidents and as such often are fractured during such trauma and may be subject to complex devastating fractures.

Mechanical devices most commonly in the form of pins, plates and screws are commonly used to attach fractured long bones. The plates, pins and screws are typically made of a durable material compatible with the human anatomy, for example titanium, stainless steel or cobalt chrome. The plates are typically positioned longitudinally along the periphery of the long bone and have holes or openings through which screws may be inserted into the long bone transversely. Additionally, intramedullary nails or screws may be utilized to secure fractured components of a long bone, for example, to secure the head of a femur.

Proper securement of a bone plate to a bone is dependent on, among other things, the condition of the bone. For example, if the bone is severely fractured, the fasteners are preferably non-locking or not rigidly secured to the plate. By not locking the fastener to the plate, the fastener can be used to pull or draw the fragments of the fractured bone together to assist in blood flow and the healing of the fracture site. Such non-locking fasteners may include, for example, fasteners with cancellous threads to securely contain the fragments. Non-locking fasteners may also include a portion of the stem, which is not threaded, or may be in the form of a lagging screw to assist in the drawing of bone fragments together. Further, the use of a non-locking fastener results in increased flexion on motion between the fasteners and the plate thereby increasing the stress or load on the fracture site. Such increase in fracture load or bracing of the stress adjacent to the fracture site results in hypertrophy or the increase in size of the cortical bone due to the physical activity to accommodate the higher stress. Such a reaction to the increased stress at the fracture site is well borne out by Wolff's Law.

Locking fasteners, for example, locking screws, provide for a more rigid construction and may provide an alternate construction for a bone plate and may be used in bone of any quality. For example, if the bone of the patient is osteoporotic or has a thin cortical layer or an eggshell cortical layer, the increased stress due to flexion between the fasteners and the bone plate caused by movable or unlocked fasteners, may fracture the cortical bone and not support such a construction. Thus, for osteoporotic bone, the use of fasteners locked to the bone plate is preferred. While x-rays and other analytical tools may be utilized to determine the type of bone of the patient, the actual condition of the bone of the patient may not be fully determined until the fracture site is exposed. Thus, there is a need to intraoperatively provide a plate, which may be selectively locked or unlocked with respect to its fasteners.

Compression of the bone at the fracture site may be desired when using bone plates. Compression can be a useful procedure to pull larger fragments in line and to encourage a faster rate of healing. Compression is particularly well suited to correct fractures in which the fractures are highly comminuted or have a large number of fragments. The compression of the bone is typically accomplished by first securing the bone plate to a position spaced from the fracture site and compressing the bone as the plate is secured at a position spaced from the fracture site and opposed to the first anchored position.

Compression of the bone at the fracture site may be accomplished through the use of a bone plate that includes dynamizing features. Dynamizing allows bone segments or fragments to be moved towards each other along the longitudinal axis of the bone plate to effect compression of the bone segments or fragments. Commonly available bone plates that provide for dynamizing require the ability to move the bone screw in a direction parallel to the longitudinal axis of the plate. Accordingly, such dynamizing systems utilize non-locking bone screws. In addition, while allowing for compression at the fracture site, such commonly available bone plate systems do not provide for distraction of the bone fragments.

While the prior art has addressed the need for locked bone plates and for dynamizing bone plates, a need remains for a bone plating system that allows for both locking a bone screw to a bone plate and for dynamizing the position of the locked bone screw and that allows for both distraction and compression of the bone segments at the fracture site.

### SUMMARY OF THE INVENTION

The present invention provides a bone plate system that allows for both locking a bone screw to the bone plate and dynamizing the position of the locked bone screw to allow for distraction of the bone segments at a bone fracture site and for applying compression to the bone segments at the bone fracture site.

In one aspect, the present invention provides a bone plate system comprising a plurality of bone screws and a bone plate. The bone plate comprises a body portion and a movable portion. The body portion has a longitudinal axis, a transverse axis and a bone screw hole having a longitudinal dimension and a transverse dimension. The body portion also has a first portion and a second portion connected to the first portion. The first and second portions are spaced apart longitudinally. The longitudinal spacing between the first and second portions is greater than the longitudinal dimension of the bone screw hole. The movable portion is in the space between the first portion and second portion of the body portion. The movable portion has a top surface, an opposite bone-facing surface, a longitudinal dimension, a transverse dimension and an interior edge defining a bone screw hole extending from the top surface to the bone-facing surface. The longitudinal dimension of the movable portion is less than the longitudinal spacing between the first portion and second portion of the body portion. The movable portion is connected to the first portion and the second portion of the body portion and is movable along the longitudinal axis of the body portion toward and away from the first portion and the second portion of the body portion. A majority of the top surface and a majority of the bone-facing surface of the movable portion are exposed in the space between the first portion and the second portion of the body portion.

In another aspect, the present invention provides a bone plate system comprising a plurality of bone screws and a bone plate. The bone screws have threaded shafts and non-threaded heads. The bone plate has first and second ends, a longitudinal axis and a transverse axis. The bone plate comprises a first end portion and a second end portion. The first and second portions each have a bone screw hole. The second end portion is spaced from the first end portion, and the two end portions are aligned along the longitudinal axis of the bone plate. The bone plate also includes a central portion spaced from the first end portion and the second end portion by longitudinal distances. The central portion is aligned with the first and second end portions along the longitudinal axis of the bone plate. A first movable portion is positioned between the first end portion and the central portion and is longitudinally aligned with the first end portion and the central portion. The first movable portion has a top surface, a bottom surface, a longitudinal dimension, an interior edge defining a bone screw hole and an undercut defining a groove at the bone screw hole between the top and bottom surfaces. The longitudinal dimension of the first movable portion is less than the longitudinal distance between the first end portion and the central portion of the bone plate. The first movable portion is movable in a longitudinal direction toward the central portion and in a longitudinal direction away from the central portion. A second movable portion is positioned between the second end portion and the central portion and is longitudinally aligned with the second end portion and the central portion. The second movable portion has a top surface, a bottom surface, a longitudinal dimension, an interior edge defining a bone screw hole and an undercut defining a groove at the bone screw hole between the top and bottom surfaces. The longitudinal dimension of the second movable portion is less than the longitudinal distance between the second end portion and the central portion of the bone plate. The second movable portion is movable in a longitudinal direction toward the central portion and in a longitudinal direction away from the central portion. The system further comprises a first radially compressible split collet having an outer surface, an inner surface defining an axial opening and tabs extending radially outward from the outer surface. The tabs are sized and shaped to be receivable in the groove at the bone screw hole of the first movable portion of the bone plate to lock the position of the first collet with respect to the first movable portion of the bone plate along an axis perpendicular to the longitudinal and transverse axes of the bone plate. The inner surface of the first collet is sized and shaped to receive the head of one of the bone screws and to frictionally engage a portion of the bone screw when radially compressed. The outer surface of the first collet is sized and shaped to be received within the bone screw hole of the first movable portion of the bone plate and to engage the first movable portion of the bone plate at the bone screw hole. The system further comprises a second radially compressible split collet having an outer surface, an inner surface defining an axial opening and tabs extending radially outward from the outer surface. These tabs are sized and shaped to be receivable in the groove at the bone screw hole of the second movable portion of the bone plate to lock the position of the second collet with respect to the second movable portion of the bone plate along an axis perpendicular to the longitudinal and transverse axes of the bone plate. The inner surface of the second collet is sized and shaped to receive the head of one of the bone screws and to frictionally engage a portion of the bone screw when radially compressed. The outer surface of the second collet is sized and shaped to be received within the bone screw hole of the second movable portion of the bone plate and to engage the second movable portion of the bone plate at the bone screw hole. In another aspect, the present invention provides a bone plate system comprising a plurality of bone screws, a bone plate and a radially compressible collet assembly. The bone screws have threaded shafts and non-threaded heads; the heads of the screws are non-threaded. The bone plate has first and second ends, a longitudinal axis, a transverse axis, a top surface, a bottom surface, an interior surface defining a bone screw hole extending from the top surface to the bottom surface, and an undercut defining a groove at the bone screw hole between the top and bottom surfaces. The radially compressible collet assembly includes an annular metal outer member and an annular non-metallic inner member. The outer member has an outer surface and tabs extending radially outward from the outer surface. The tabs are sized and shaped to be receivable in the groove at the bone screw hole of the bone plate to lock the position of the collet assembly with respect to the bone plate along an axis perpendicular to the longitudinal and transverse axes of the bone plate. The inner member has an inner surface defining an axial opening. The inner surface of the inner member is sized and shaped to receive the head of one of the bone screws and to frictionally engage a portion of the bone screw when radially compressed. The outer surface of the outer member is sized and shaped to be received within the bone screw hole of the bone plate and to engage the interior surface of the bone plate. The inner member and the outer member have spaced axial slots and concentric portions. The concentric portions are sized and shaped so that the concentric portion of the outer member is radially compressible by the interior surface of the bone plate and so that the concentric portion of the inner member is radially compressible by radial compression of the outer member. The inner member and bone screw are sized and shaped so that radial compression of the inner member causes the inner member to engage the bone screw to limit movement of the bone screw with respect to the collet assembly. The collet assembly has a first end at the annular outer member, a second end at the annular inner member and an axial dimension between the first end and the second end. The distance between the top surface and the bottom surface of the bone plate is at least equal to the axial dimension of the collet assembly.

### BRIEF DESCRIPTION OF THE DRAWINGS

The invention will be better understood by reference to the figures of the drawings wherein like numbers denote like parts throughout and wherein:

FIG. 1 is a top plan view of an embodiment of a bone plate that may be used with the bone plate system of the present invention;

FIG. 2 is a cross-section of one of the movable portions of the bone plate of FIG. 1, taken along line 2-2 of FIG. 1;

FIG. 3 is an end view of the bone plate of FIG. 1, taken along line 3-3 of FIG. 1;

FIG. 4 is an elevation of a set of bone screws and locking bone screw assemblies that may be used in the bone plate system of the present invention;

FIG. 5 is a top plan view of an alternative embodiment of a bone plate that may be used in the bone plate system of the present invention;

FIG. 6 is an end view of the bone plate of FIG. 5, taken along line 6-6 of FIG. 5;

FIG. 7 is a cross-section of one of the movable portions of the bone plate of FIG. 6, taken along line 7-7 of FIG. 6;

FIG. 7A is a representative longitudinal cross-section of one of the threaded bores of the movable portions of the bone plate;

FIG. 8 is a top plan view of another alternative embodiment of a bone plate that may be used in the bone plate system of the present invention;

FIG. 9 is a cross-section of one of the end portions of the bone plate of FIG. 8, taken along line 9-9 of FIG. 8;

FIG. 10 is a cross-section of one of the movable portions of the bone plate of FIG. 8, taken along line 10-10 of FIG. 8;

FIG. 11 is a partial cross-section of one of the locking bone screw assemblies of FIG. 4, showing the collet of the assembly in cross-section and the bone screw in elevation;

FIG. 12 is a perspective view of the outer member of the collet of FIG. 8;

FIG. 13 is a side elevation of the outer member of FIG. 12;

FIG. 14 is top plan view of the outer member of FIGS. 12-13;

FIG. 15 is a cross-section of the outer member of FIGS. 12-14, taken along line 12-12 of FIG. 11;

FIG. 16 is a bottom plan view of the outer member of FIGS. 12-15;

FIG. 17 is a perspective view of the inner member of the collet of FIG. 11;

FIG. 18 is a side elevation of the inner member of FIG. 17;

FIG. 19 is a top plan view of the inner member of FIGS. 17-18;

FIG. 20 is a cross-section of the inner member of FIGS. 17-19, taken along line 20-20 of FIG. 19;

FIG. 21 is a bottom plan view of the inner member of FIGS. 17-20;

FIG. 22 shows one of the movable portions of the bone plate in longitudinal cross-section in place on a bone segment, also shown in cross-section, shown with one of the locking bone screw assemblies inserted through the bone screw hole of the movable portion of the bone plate with the collet shown in longitudinal cross-section and the bone screw shown in elevation;

FIG. 23 is a view similar to FIG. 22, showing the bone screw advanced to a position below the top surface of the movable portion of the bone plate;

FIG. 24 is a view similar to FIGS. 22-23, showing the locking bone screw assembly fully seated with respect to the bone plate for locking the position of the bone screw assembly with respect to the bone plate, and showing a partial view of a tool that may be used to seat the locking bone screw assembly;

FIG. 25 is a view similar to FIG. 22, showing use of an alternative collet;

FIG. 26 is a view similar to FIG. 24, showing the locking bone screw assembly of FIG. 25 fully seated with respect to the movable portion of the bone plate.

### DETAILED DESCRIPTION OF THE ILLUSTRATED EMBODIMENTS

Three embodiments of bone plate systems illustrating the principles of the present invention are illustrated in the accompanying drawings. The illustrated embodiments of the systems are locking plates, in which at least some of the bone screws are locked to the bone plate. Locking plates are advantageous in that motion (including micromotion) between the plate and the bone screws is minimized or eliminated to prevent loosening of the connection between the bone plate and the bone segments. The illustrated embodiments provide the advantage of locking plates while also allowing for dynamizing the positions of the bone screws. Dynamization is the movement of bone screws in a direction parallel to the longitudinal axis of the plate. Dynamization is essential for compression of the fracture site being treated. The embodiments of the present invention also allow for distraction of the fracture site. The embodiments of the system of the present invention may be substantially preassembled to avoid difficult, time-consuming intraoperative assembly procedures.

The bone plating system of the present invention includes a bone plate, a plurality of bone screws, and one or more collets. Each collet may be preassembled with one bone screw for use with the system. As described in more detail below, the bone plating system of the present invention may also include a tamping instrument for locking the collet-bone screw assembly to the bone plate.

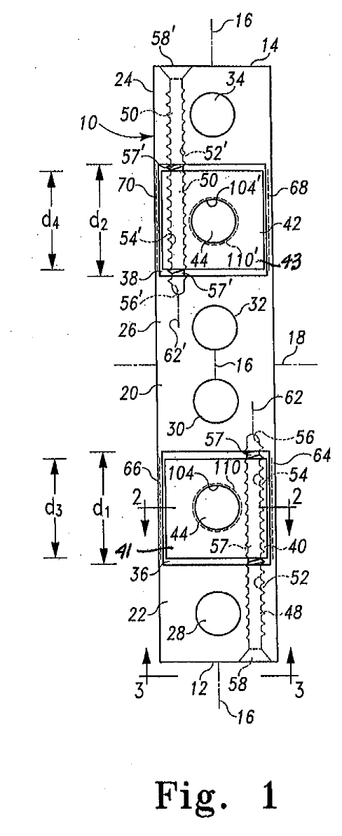

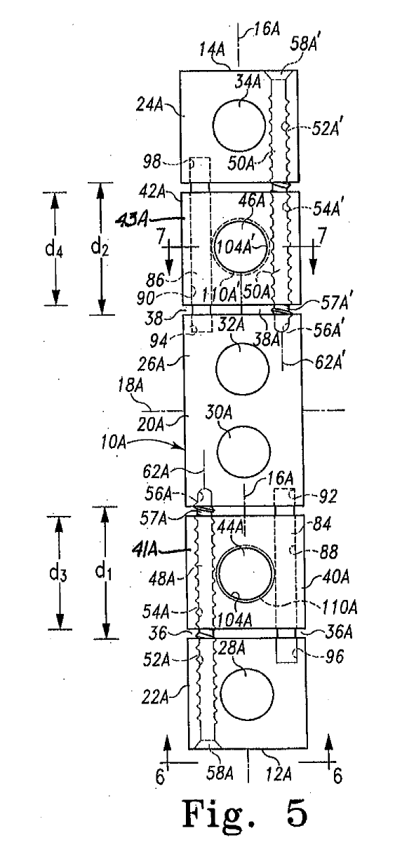

A first example of a bone plate that may be used in the bone plating system of the present invention is illustrated in FIG. 1 at 10. A second example of a bone plate that may be used in the bone plating system of the present invention is illustrated in FIG. 5 at 10A. A third example of a bone plate that may be used in the bone plating system of the present invention is illustrated in FIG. 8 at 10B. In the following description and in the drawings, similar parts of the first, second and third embodiments are designated by like reference numbers, followed by the suffix “A” for the embodiment of FIG. 5 and the suffix “B” for the embodiment of FIG. 8.

Referring now to FIGS. 1-3, the first illustrated bone plate 10 has a first end 12, a second end 14, a longitudinal axis 16, a transverse axis 18 and a fixed body portion 20. The body portion 20 includes a first end portion 22, a second end portion 24 and a central portion 26. These three portions 22, 24, 26 are all aligned along the longitudinal axis 16 of the bone plate and fixed with respect to each other. All three of these portions 22, 24, 26 have bone plate holes 28, 30, 32, 34.

The first end portion 22 is spaced from the central portion 26 by a fixed longitudinal distance, shown at d₁ in FIG. 1, to define an enlarged opening or space 36 between the first end portion 22 and the central portion 26. The second end portion 24 is spaced from the central portion 26 by a fixed longitudinal distance, shown at d₂ in FIG. 1, to define a second enlarged opening or space 38 between the second end portion 24 and the central portion 26.

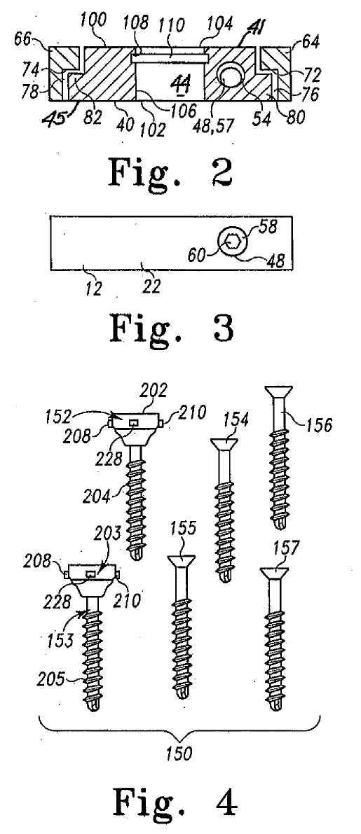

The bone plate 10 also includes a first movable portion 40 and a second movable portion 42. The first movable portion 40 is positioned in the first enlarged opening or space 36 between the first end portion 22 and the central portion 26. The second movable portion 42 is positioned in the second enlarged opening or space 38 between the second end portion 24 and the central portion 26. Both movable portions 40, 42 are aligned along the longitudinal axis 16 of the bone plate 10 with the end portions 22, 24 and central portion 26 of the bone plate 10. Each movable portion 40, 42 has a bone screw hole 44, 46 extending from a top surface 41, 43 to an opposite bone-facing surface 45 (shown in FIG. 2 for the first movable portion 40). The longitudinal dimension of the first movable portion 40, illustrated at d₃ in FIG. 1, is less than the longitudinal spacing (shown at d₁) between the first end portion 22 and central portion 26, of the enlarged opening or space 36.

As described in more detail below, the difference between d₁ and d₃ represents the potential longitudinal distance that the first movable portion 40 can travel toward and away from the central portion 26. Similarly, the longitudinal dimension of the second movable portion 42, illustrated at d₄ in FIG. 1, is less than the longitudinal spacing (shown at d₂) between the second end portion and the central portion. The difference between d₂ and d₄ represents the potential longitudinal distance that the second movable portion 42 can travel toward and away from the central portion 26. The total of these two differences represents the total possible distance available for distraction and compression of bone fragments or segments to which the bone plate 10 is attached. By way of example, the components of the plate may be sized so that each movable portion 40, 42 can travel about 2 mm in either direction from the positions shown in FIG. 1, thus allowing for total travel of about 4 mm for each movable portion, and a total travel of about 8 mm for both movable portions; it should be understood that this dimension is provided as an example only, and that the invention is not limited to any particular dimension unless expressly called for in the claims.

To allow the movable portions 40, 42 to be moved longitudinally in the openings or spaces 36, 38 toward and away from the central portion 26, linear drive mechanisms are provided in the first illustrated bone plate 10. In the embodiment of FIGS. 1-2, the linear drive mechanisms comprise a first drive spindle mechanism 48 and a second drive spindle mechanism 50. The first drive spindle mechanism 48 comprises an elongate straight shaft extending from the first end 12 of the bone plate 10 through a smooth bore 52 (shown in phantom in FIG. 1) in the first end portion 22, through a threaded bore 54 (shown in phantom in FIG. 1) in the first movable portion 40 and terminates in a smooth blind bore 56 (shown in phantom in FIG. 1) in the central portion 26 of the bone plate. At least part of the length of the first drive spindle mechanism 48 is threaded (as shown at 57 in FIG. 1) and engages the threaded bore 54 of the first movable portion 40. The portions of the shaft of the first drive spindle mechanism 48 received in the smooth bores 52, 56 may be threaded or smooth. As illustrated, the bores 52, 54, 56 are all co-axially aligned along a longitudinal axis. The first spindle drive mechanism 48 has a head 58 (shown in FIG. 3) exposed at the first end 12 of the bone plate, and may include any standard shape recess 60 to receive a tool (not shown) for turning the spindle drive mechanism 48 about its longitudinal axis 62. The second end portion 24, second movable portion 42 and second spindle drive mechanism 50 have similar features, identified at 52′, 54′ and 57′ in FIG. 1.

It will be appreciated that the number of threads per inch for the spindle drive mechanisms 48, 50 and the pitch of the threads can be selected to provide the desired level of translational movement for each turn of the spindle.

As shown in FIG. 1, the first and second movable portions 40, 42 of the first illustrated bone plate 10 are bordered by spaced longitudinal sides 64, 66, 68, 70. One pair of spaced longitudinal sides 64, 66 extend between and connect the first end portion 22 and the central portion 26 of the bone plate. The other pair of spaced longitudinal sides 68, 70 extend between and connect the second end portion 24 and the central portion 26 of the bone plate 10.

As illustrated in FIG. 2, spaced longitudinal sides 64, 66 have undercuts 72, 74 defining channels 76, 78 that receive complementary longitudinal flanges 80, 82 on the first movable portion 40. Although the flanges 80, 82 may slide in the channels 76, 78, the complementary structures of the channels and flanges serve to control movement of the movable portion 40 so that such movement is limited to longitudinal movement. It should be understood that longitudinal flanges could be provided on the longitudinal sides to be received in longitudinal channels in the movable portion 40. The longitudinal sides 68, 70 and second movable portion 42 in the first illustrated bone plate have similar structures to constrain movement of the second movable portion 42 to longitudinal linear movement.

The bone plate 10A illustrated in FIGS. 5-7 shares several similar features with the bone plate 10 of FIGS. 1-3. The bone plate 10A has a first end 12A, second end 14A, longitudinal axis 16A, transverse axis 18A, fixed body portion 20A, first end portion 22A, second end portion 24A, central portion 26A, bone plate holes 28A, 30A, 32A, 34A, enlarged openings or spaces 36A, 38A, first and second movable portions 40A, 42A, bone screw holes 44A, 46A, first and second drive spindle mechanisms 48A, 50A with heads 58A, 58A′, recesses 60A, longitudinal axes 62A, 62A′, smooth bores 52A, 52A′, threaded bores 54A, 54A′ and smooth blind bores 56A, 56A′ substantially the same as described above for the bone plate 10 of FIGS. 1-3. Distances d₁, d₂, d₃ and d₄ may be the same as those for the embodiment of FIGS. 1-3.

The second illustrated bone plate 10A differs from the first illustrated bone plate 10 in that the second illustrated bone plate 10A does not include longitudinal sides 64, 66, 68, 70, undercuts 72, 74, channels 76, 78 or flanges 80, 82. Instead, movement of the first and second movable portions 40A, 42A of the second illustrated bone plate 10A is constrained by a set of guide rods 84, 86 extending longitudinally outward from the first and second end portions 22A, 24A toward the central portion 26A. These guide rods 84, 86 extend through smooth bores 88, 90 in the first and second movable portions 40A, 42A and have ends received in smooth blind bores 92, 94 in the central portion 26A of the bone plate 10A and in smooth blind bores 96, 98 in the end portions 22A, 24A. The first and second movable portions 40A, 42A may slide on the guide rods 84, 86, and the guide rods 84, 86 serve to constrain movement of the first and second movable portions 40A, 42A to linear longitudinal paths of travel.

Thus, with both embodiments 10, 10A, rotation of the spindle drive mechanisms 48, 48A, 50, 50A in one direction causes longitudinal translational movement of the movable portions 40, 40A, 42, 42A toward the central portion 26, 26A and rotation of the spindle drive mechanisms 48, 48A, 50, 50A in the opposite direction causes longitudinal translational movement of the movable portions 40, 40A, 42, 42A away from the central portion 26, 26A. When the movable portions 40, 40A, 42, 42A are fixed to bone segments or fragments, longitudinal translational movement of the movable portions 40, 40A, 42, 42A away from the central portion 26, 26A effects distraction of the bone segments of fragments and longitudinal translational movement of the movable portions 40, 40A, 42, 42A toward the central portion 26, 26A effects compression of the bone segments or fragments.

The bone plate 10B illustrated in FIGS. 8-10 shares several similar features with the bone plate 10 of FIGS. 1-3 and bone plate 10A of FIGS. 5-7. The bone plate 10B has a first end 12B, second end 14B, longitudinal axis 16B, transverse axis 18B, fixed body portion 20B, first end portion 22B, second end portion 24B, central portion 26B, bone plate holes 28B, 30B, 32B, 34B, enlarged openings or spaces 36B, 38B, first and second movable portions 40B, 42B, and bone screw holes 44B, 46B, similar to those described above for the bone plate 10 of FIGS. 1-3 and bone plate 10A of FIGS. 5-7. Distances d₁, d₂, d₃ and d₄ may be the same as those for the embodiments of FIGS. 1-3 and 5-7.

However, in the third illustrated bone plate 10B, the plate 10B does not include any drive mechanism. Instead, the movable portions 40B, 42B are positioned in the enlarged openings or spaces 36B, 38B of body portion 20B, and are mounted to the body portion 20B in a manner that allows the movable portions 40B, 42B to move freely along the longitudinal axis 16B of the bone plate 10B.

In the third illustrated embodiment, the movable portions 40B, 42B are mounted on rails 83B, 85B, 87B, 89B that extend longitudinally across the enlarged openings or spaces 36B, 38B. The ends of the rails 83B, 85B, 87B, 89B are received in smooth blind bores 91B, 92B, 93B, 94B, 95B, 96B, 97B, 98B formed in the first and second end portions 22B, 24B and the central portion 26B. These rails 83B, 85B, 87B, 89B extend through smooth bores 88B, 90B, 99B, 101B in the first and second movable portions 40B, 42B and into the blind bores 91B, 92B, 93B, 94B, 95B, 96B, 97B, 98B. The first and second movable portions 40B, 42B may slide on the rails 83B, 85B, 87B, 89B; the rails serve to constrain movement of the first and second movable portions 40B, 42B to linear longitudinal paths of travel, while allowing for free longitudinal movement in the confines of the enlarged openings or spaces 36B, 38B. The illustrated rails 83B, 85B, 87B, 89B and the smooth bores 88B, 90B, 99B, 101B and blind bores 91B, 92B, 93B, 94B, 95B, 96B, 97B, 98B are square in cross-section, although it should be understood that the rails and bores could have other shapes, such as circular in cross-section. It should also be appreciated that structures other than rails can be used to mount the movable portions 40B, 42B to the body portion 20B in a way that allows the movable portions 40B, 42B to slide along the longitudinal axis 16B of the body portion 20B.

When the third illustrated plate 10B is implanted in a patient, the patient's body weight (an applied axial load) dynamically loads the plate 10B because part of the plate could slide in relation to the rest of the plate. Thus, with relative longitudinal movement between the movable portions 40B, 42B and the body portion 20B being allowed post-implantation, the third illustrated plate 10B provides a dynamic locking plate.

To allow for fixation of the movable portions 40, 40A, 42, 42A of the bone plate 10 to the bone segments or fragments, the bone screw holes 44, 44A, 46, 46A allow for a locked connection between the movable portions 40, 40A, 42, 42A and bone screw assemblies, described in more detail below. Although in the illustrated embodiments, each movable portion 40, 40A, 42, 42A has a single bone screw hole 44, 44A, 46, 46A, it should be understood that additional bone screw holes could be provided in the movable portions 40, 40A, 42, 42A if desired. Such additional bone screw holes could have the structure described below for holes 44, 44A, 46, 46A or could have the structure described below for holes 28, 28A, 30, 30A, 32, 32A, 34, 34A.

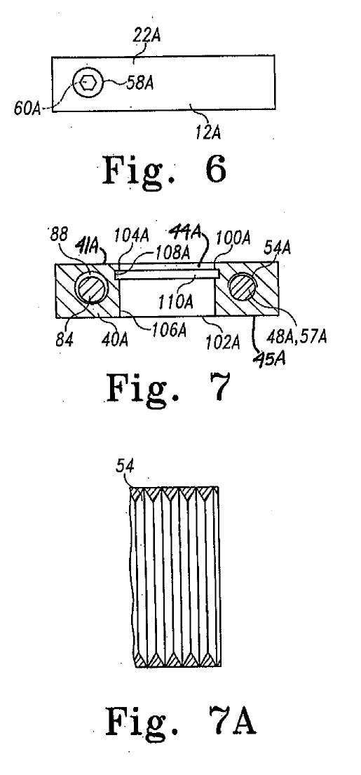

FIG. 2 illustrates a cross-section of one of the movable portions 40 of the bone plate 10; FIG. 6 illustrates a cross-section of one of the movable portions 40A of the bone plate 10A; and FIG. 10 illustrates a cross-section of one of the movable portions 40B of the bone plate 10B. It should be understood that the second movable portion 42, 42A, 42B may have the same structure as described below. The movable portion 40, 40A, 40B of the bone plate 10, 10A, 10B has a top surface 100, 100A, 100B and a bottom surface 102, 102A, 102B. The bone screw hole 44, 44A, 44B is defined by a circular interior edge 104, 104′, 104A, 104A′, 104B, 104B′ (shown in FIGS. 1, 5 and 8) and a cylindrical interior surface 106, 106A, 106B (shown in FIGS. 2, 7 and 10) and extends from the top surface 100, 100A, 100B to the bottom surface 102, 102A, 102B. Near the top surface 100, 100A, 100B the movable portion 40, 42, 40A, 42A, 40B, 42B has an undercut 108, 108A, 108B (shown in FIGS. 2, 7 and 10) defining a groove 110, 110A, 110B (shown in FIGS. 2, 7 and 10) in the cylindrical interior surface 106, 106A, 106B. The illustrated groove 110, 110A, 110B extends around the circumference of the bone screw hole 44, 44A, 44B, although it should be understood that two or more grooves defined by arcs could be provided. It should also be understood that although a circular opening defines the illustrated bone screw hole 44, 44A, 44B other shapes may be used, such as a longitudinally elongated slot, for example. As described in more detail below, the groove 110, 110A, 110B is provided to receive tabs on a locking bone screw assembly to lock the movable portion 40, 40A, 40B, 42, 42A, 42B and the locking bone screw assembly 152 together. The interior edge of the bone screw hole 46, 46A, 46B of the second movable portion 42, 42A, 42B is shown at 104′, 104A′, 104B′ and the groove of the bone screw hole 46, 46A, 46B is shown at 110′, 110A′, 110B′ in FIGS. 1, 5 and 8 and have the structure described above.

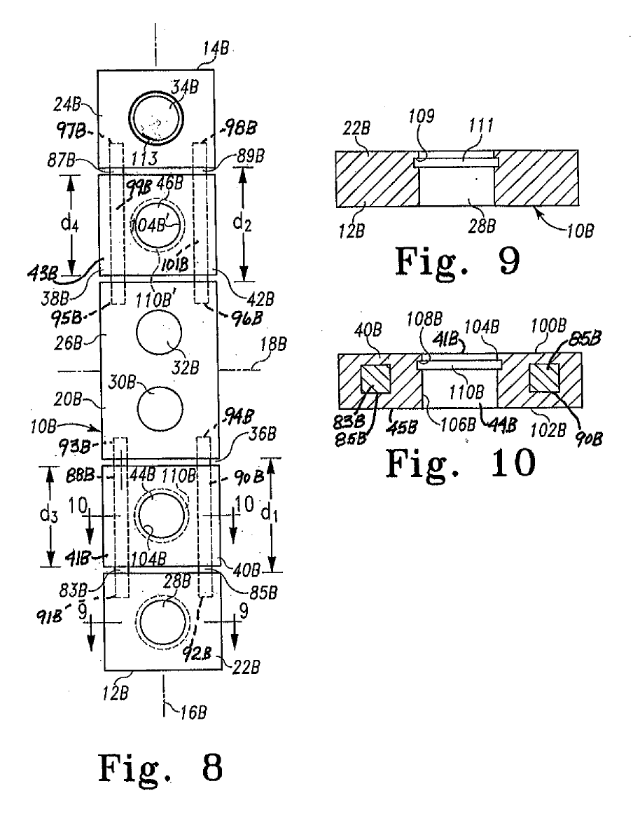

The bone plate holes 28, 28A, 28B, 30, 30A, 30B, 32, 32A, 30B, 34, 34A, 34B of the fixed portions 22, 22A, 22B, 24, 24A, 24B, 26, 26A, 26B of the bone plates 10, 10A, 10B in the illustrated embodiments are defined by circular interior edges and cylindrical interior surfaces. Any or all of these bone plate holes 28, 28A, 28B, 30, 30A, 30B, 32, 32A, 32B, 34, 34A, 34B may have undercuts and grooves of the type described above for the holes 44, 44A, 44B, 46, 46A, 46B to allow for these holes to be used with a locking bone screw assembly, or may not include such undercuts and grooves. The embodiment of FIGS. 8-9 illustrates such an undercut 109 and groove 111 for the hole 28B in the fixed portion 22B. If such undercuts and grooves are provided for these holes 28, 28A, 28B, 30, 30A, 30B, 32, 32A, 32B, 34, 34A, 34B, the surgeon has several options available for screws to be used in these holes: the surgeon may opt to use a locking bone screw or bone screw assembly; or the surgeon may opt to use a non-locking bone screw or bone screw assembly. The top surfaces 100, 100A, 100B of the bone plates may also have annular countersinks surrounding the screw holes 28, 28A, 28B, 30, 30A, 30B, 32, 32A, 32B, 34, 34A, 34B for recessing of the bone screw heads. An example of such a countersink is illustrated at 113 in FIG. 8 surrounding hole 34B in the fixed portion 24B; it should be understood that this position of the countersink is illustrative only; such a countersink could be provided surrounding any of the screw holes 28, 28A, 28B, 30, 30A, 30B, 32, 32A, 32B, 34, 34A, 34B in the illustrated embodiments.

The illustrated bone plates 10, 10A, 10B may be made of any suitable durable material that is biologically compatible with the human anatomy and preferable made of a high strength metal. For example, the plate may be made of stainless steel, cobalt chrome or titanium. A forged or wrought titanium alloy may be used, such as ASTM F-620-97 or ASTM F-136 ELI.

Although the illustrated bone plates 10, 10A, 10B are elongated structures of substantially constant width along their lengths, the end portions may have other shapes. For example, one of the end portions may form an enlarged head suitable for fixation to the end of a bone, such as the distal femur or proximal tibia.

The illustrated bone plates 10, 10A, 10B may comprise part of a bone plate system that includes a plurality of bone screws. An example of a set of bone screws that could be used with the system of the present invention is illustrated at 150 in FIG. 4. The illustrated set of bone screws 150 includes both locking bone screw assemblies 152, 153 and non-locking bone screws 154, 155, 156, 157. It should be appreciated that the set of bone screws could include polyaxial bone screw or bone peg assemblies, such as those disclosed in U.S. Patent Publication No. 2005/0187551 A1 entitled “Bone Plate System with Bone Screws Fixed by Secondary Compression,” filed by Orbay et al., the disclosure of which is incorporated by reference herein in its entirety. In addition, the bone plate assembly could be used with anchor elements having spherical heads and receiving structures and caps of the type disclosed in U.S. Pat. No. 7,087,057 entitled “Polyaxial Bone Screw”, the disclosure of which is incorporated by reference herein in its entirety. Such anchor elements, receiving structures and caps may be sized and shaped to fit within the bone plate holes 28, 28A, 28B, 30, 30A, 30B, 32, 32A, 32B, 34, 34A, 34B.

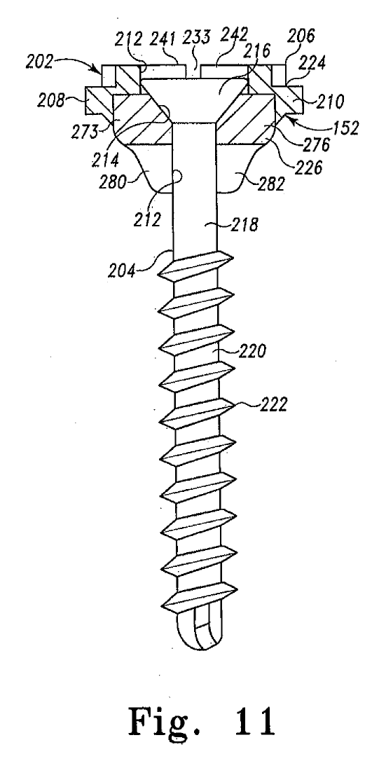

As shown in FIG. 4, the locking bone screw assemblies 152, 153 of the present invention include first and second collets 202, 203 and bone screws 204, 205. A representative locking bone screw assembly 152 is illustrated in FIG. 11 with the collet 202 shown in cross-section. The collet 202 is generally annular and includes an outer surface 206 with diametrically-opposed tabs 208, 210 extending radially outward from the outer surface 206. The tabs 208, 210 are sized and shaped to be receivable in the grooves 110, 110A, 110B, 110′, 110A′, 110B′ of the movable portions 40, 40A, 40B, 42, 42A, 42B of the bone plate 10, 10A, 10B. The collet 202 also includes an inner surface 212 defining an axial opening or bore 214 that is sized and shaped to receive the head 216 of the bone screw 204 and to engage a smooth portion 218 of the shank 220 of the bone screw 204. The shank 220 also includes a threaded portion 222.

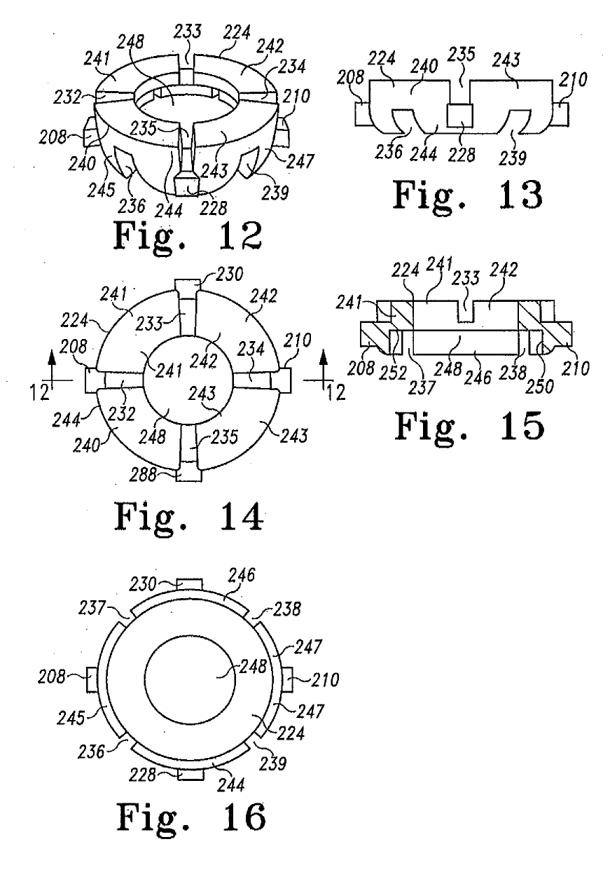

The illustrated collet 202 comprises an assembly of an annular outer member 224 and an annular inner member 226. An example of a suitable structure for the annular outer member 224 is illustrated in FIGS. 12-16. As shown in FIGS. 14 and 16, the illustrated annular outer member 224 includes two additional tabs 228, 230 not visible in the cross-section of FIG. 11. The four tabs 208, 210, 228, 230 are spaced evenly about the circumference of the annular outer member 224. The annular outer member 224 has a plurality of slots 232, 233, 234, 235 (see FIGS. 12 and 14) extending both axially and radially and slots 236, 237, 238, 239 (see FIG. 16) extending both axially and radially. The slots 232, 233, 234, 235, 236, 237, 238, 239 define radially compressible fingers 240, 241, 242, 243 (see FIGS. 12 and 14) and radially compressible fingers 244, 245, 246, 247 (see FIG. 16). As shown in FIG. 15, the annular outer member 224 has an inner bore 248, an inner cylindrical surface 250 and an inner radial surface 252 adjoining the cylindrical surface 250. The annular outer member 224 may be made of metal, such as stainless steel, a standard cobalt -chrome alloy or a standard titanium alloy used in implantable medical devices, for example. One suitable alloy is ASTM F-620-97 and another suitable alloy is ASTM F-136 ELI. However, it should be understood that the present invention is not limited to any particular material unless expressly called for in the claims.

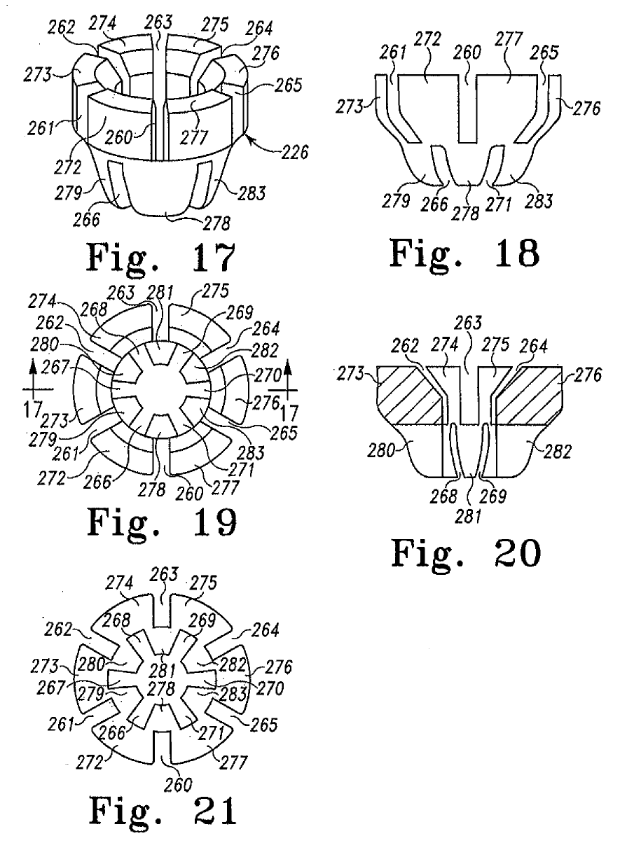

An example of a suitable structure for the annular inner member 226 is illustrated in FIGS. 17-21. The annular inner member 226 has a plurality of slots 260, 261, 262, 263, 264, 265 (see FIGS. 17 and 19) extending axially and radially and slots 266, 267, 268, 269, 270, 271 (see FIG. 19) extending axially and radially. The slots 260, 261, 262, 263, 264, 265, 266, 267, 268, 269, 270, 271 define a plurality of radially compressible fingers 272, 273, 274, 275, 276, 277 (see FIGS. 17, 19 and 21) and radially compressible fingers 278, 279, 280, 281, 282, 283 (see FIG. 19 and 21). The annular inner member 226 may be made of a non-metallic material, such as of a standard medical grade polymer, such as ultrahigh molecular weight polyethylene, for example. A biocompatible, biostable, implantable medical grade elastomeric material, such as elastomeric thermoplastic polymers (e.g. medical grade polyurethanes) or silicone (e.g. medical grade silicone rubber) may also be used. It should be understood that the present invention is not limited to any particular material unless expressly called for in the claims.

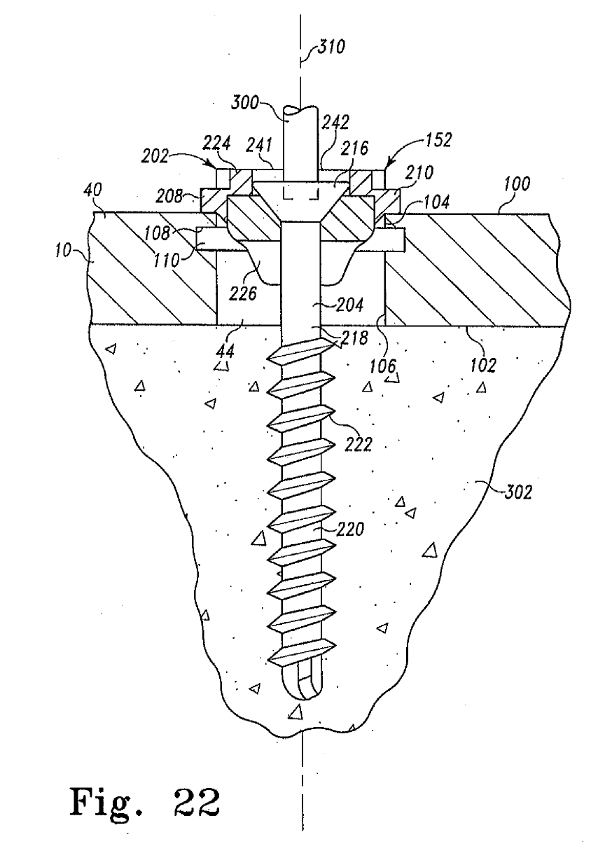

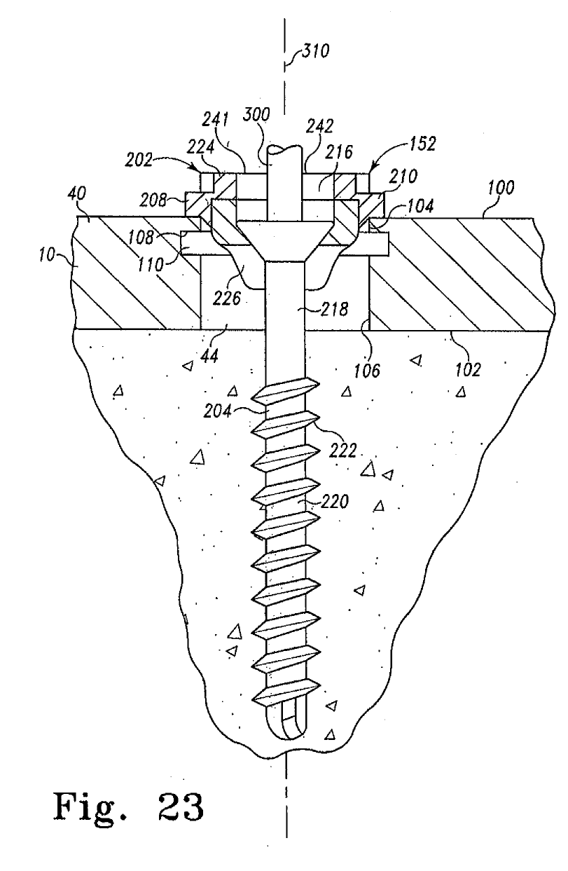

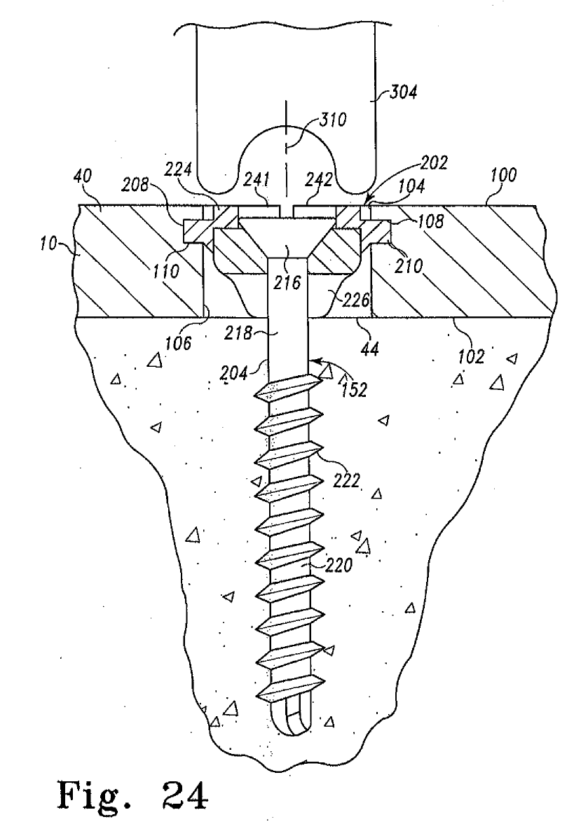

When the outer and inner annular members 224, 226 are assembled, the top surfaces and radially outer surfaces of the fingers 272, 273, 274, 275, 276, 277 of the inner member 226 contact the inner cylindrical surface 250 and an inner radial surface 252 of the outer member 224. Thus, portions of the fingers 272, 273, 274, 275, 276, 277 of the inner member 226 are concentric with portions of the fingers 244, 245, 246, 247 of the outer member 224, and are received within a portion of the outer member. Thus, the assembly of the annular outer and inner members 224, 226 defines a radially compressible split collet. When assembled with the bone screw 204 as shown in FIGS. 22-23, portions of the fingers 278, 279, 280, 281, 282, 283 of the inner member 226 engage the smooth portion 218 of the shank 220 of the bone screw 204, and portions of the fingers 272, 273, 274, 275, 276, 277 of the inner member 226 engage the head 216 of the bone screw 204.

FIG. 22 illustrates use of the locking bone screw assembly 152 with one of the movable portions 40 of the bone plate 10 of FIG. 1. As shown in FIG. 22, the assembly 152 is placed at the hole 44 with the shank 220 and part of the radially compressible split collet assembly 202 and shank 220 of the bone screw 204 extending into the hole 44 of the movable portion of the bone plate 10. The head 216 of the bone screw 204 is turned by a suitable tool, such as that shown at 300 in FIG. 22, to drive the threaded portion 222 of the shank 220 into an underlying bone segment 302 until the bottom surfaces of the tabs 208, 210 rest against the top surface 100 of the movable portion 40 of the bone plate 10.

As shown in FIG. 23, the head 216 the bone screw 204 may continue to be turned to drive the bone screw further into the bone, and may be driven into the bone until the top surface of the head 216 of the bone screw is between the top and bottom surfaces 100, 102 of the movable portion of the bone plate 10. As illustrated in FIG. 23, the inner member 226 of the collet assembly may be made of a compressible material with elastic properties so that the inner member compresses axially by the advancement of the head 216 of the bone screw.

When the bone screw is in the desired position, the surgeon then uses an impact instrument, such as that shown at 304 in FIG. 24, to impact the top surfaces of the fingers 240, 241, 242, 243 of the outer annular member 224 and drive the collet assembly 202 into the bone screw hole 44 until the tabs 208, 210, 228, 230 are received in the groove 110 in the interior surface 106 of the hole 44. Preferably, the collet assembly 202 moves along the longitudinal axis of the bone screw 204 so that the bone screw 204 does not advance any further into the bone from the impaction. When the tabs 208, 210, 228, 230 are so received in the groove 110, the outer member 224 of the collet is locked against axial movement. The interior surface 106 defining the hole 44 effects a radial compressive force that acts against the fingers 244, 245, 246, 247 of the outer member 224 and these fingers 244, 245, 246, 247 effect a radial compressive force against the fingers 272, 273, 274, 275, 276, 277 of the inner annular member 226, compressing these fingers 272, 273, 274, 275, 276, 277 against the head 216 and smooth portion 218 of the shank 220 of the screw 204. The inner member 226 may expand axially. Thus, the position of the bone screw 204 is locked with respect to the collet assembly 202, which is locked with respect to the movable portion 40 of the bone plate along both the longitudinal and transverse axes 16, 18 of the bone plate 10 as well as along an axis 310 (shown in FIGS. 22-23) perpendicular to both the longitudinal and transverse axes 16, 18. Accordingly, the bone screw 204 is locked with respect to the movable portion 40 of the bone plate 10.

It will be appreciated that the dimensions of the outer diameter of the outer member 224 of the collet assembly 202 and the inner diameter of the interior surface 106 of the hole 44 may be selected so that the outer member 224 is radially compressed by the surface 106 when the collet assembly is fully seated as in FIG. 24. Although the interior surface 106 may be cylindrical in shape, it may also be desirable to provide a taper on the interior surface 106, such as by making the interior surface 106 frustoconical or including a frustoconical section.

To use the dynamization feature of the present invention, the surgeon would repeat the process described above to lock another bone screw assembly 153 to the second movable portion 42 of the bone plate 10 and to a second segment or fragment of bone. With both movable portions 40, 42 of the bone plate 10 locked to bone screw assemblies 152, 153 that are in turn locked to separate bone segments or fragments, movement of the movable portions 40, 42 of the bone plate will effect movement of the bone segments or fragments to which they are affixed. For example, movement of the portions 40, 42 away from the central portion 26 will distract these bone segments or fragments and movement of the portions 40, 42 toward the central portion 26 will compress the bone segments or fragments. Once the surgeon is satisfied with the relative positions of the bone segments or fragments, additional bone screws may be inserted through the other available bone plate holes 28, 30, 32, 34 and into the bone segments to stabilize the fracture. These other fixation areas may be locking or non-locking and may be polyaxial if desired.

Alternatively, the surgeon may lock one bone screw assembly to one movable portion of the bone plate, such as portion 40, on one side of the fracture and may lock another bone screw assembly to either the center portion 26 or one of the end portions 22, 24 on the opposite side of the fracture through one of the holes 32, 34. The movable portion 40 may then be moved to either distract or compress the bone fragments or segment. It will be appreciated that, accordingly, a bone plate could include a single movable portion instead of two movable portions as illustrated in the accompanying drawings.

Thus, the present invention allows for both locking the bone plate to some of the bone screws and for dynamization for either distraction or compression of the bone fragments.

It should be appreciated that although it is believed to be advantageous to combine the bone plates with movable portions 40, 42 with the illustrated bone screw assemblies 152, 153, other structures for locking a bone screw to a bone plate may be used. It should also be appreciated that although the illustrated bone plate holes are circular, other shapes, such as slots, could be used in the present invention.

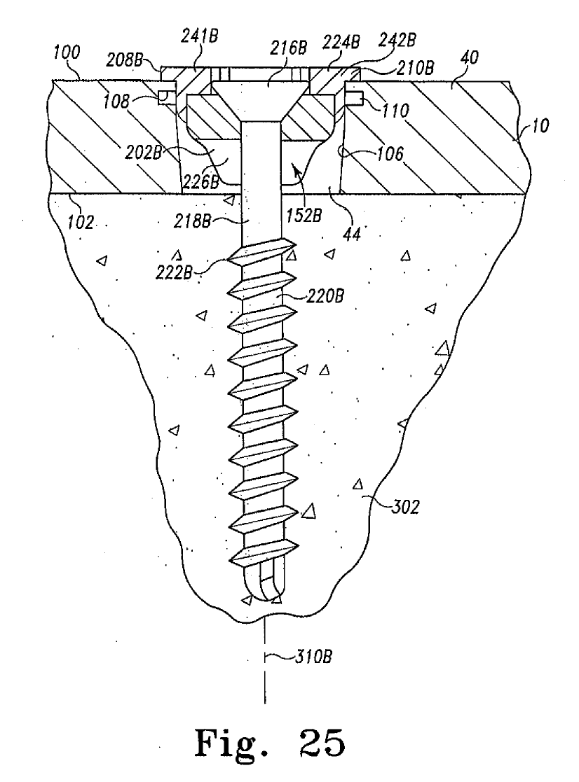

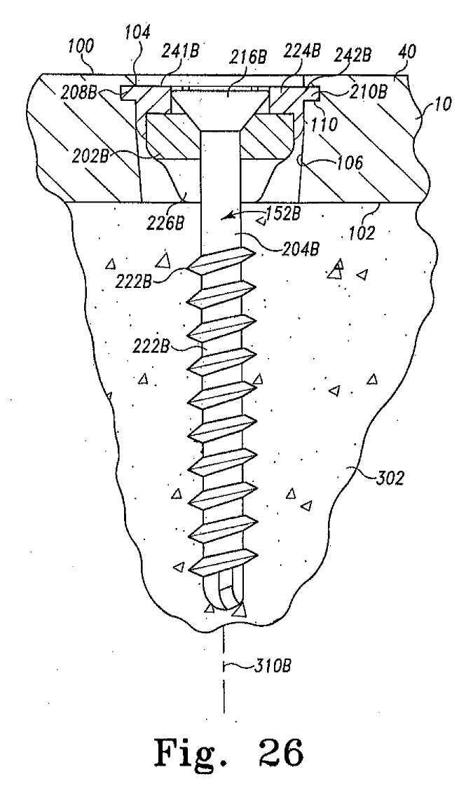

FIGS. 25-26 illustrate an additional embodiment of a bone screw assembly 152B that may be used with the movable portion 40, 40A, 40B, 42, 42A, 42B of a bone plate 10, 10A, 10B. Parts of the bone screw assembly 152 B are similar to those of FIGS. 11 and 22-24 and are labeled with the same reference numbers followed by the suffix “B”; these parts may be as described above. The embodiment of FIGS. 25-26 differs from those of FIGS. 11 and 22-24 in the placement of the tabs extending outward from the outer member 224B. In the embodiment of FIGS. 25-26, the tabs 208B, 210B extend radially outward from the upper fingers 241B, 242B at the top level of the outer member 224B. With this embodiment, the degree of relative axial movement between the collet assembly 202B and the bone screw 204B is shortened when the head 216B of the screw 204B is positioned between the top and bottom surfaces 100, 102 of the movable portion 40 of the bone plate 10.

While only specific embodiments of the invention have been described and shown, it is apparent that various alternatives and modifications can be made thereto. Those skilled in the art will also recognize that certain additions can be made to the illustrative embodiment. It is, therefore, the intention in the appended claims to cover all such alternatives, modifications and additions as may fall within the true scope of the invention.

We claim:

1. A bone plate system comprising:
a plurality of bone screws;
a bone plate comprising:
a body portion having a longitudinal axis, a transverse axis, a bone screw hole having a longitudinal dimension and a transverse dimension, the body portion having a first portion and a second portion connected to the first portion, the first and second portions being spaced apart longitudinally, the longitudinal spacing between the first and second portions being greater than the longitudinal dimension of the bone screw hole; and
a movable portion in the space between the first portion and second portion of the body portion, the movable portion having a top surface, an opposite bone-facing surface, a longitudinal dimension, a transverse dimension and an interior edge defining a bone screw hole extending from the top surface to the bone-facing surface, the longitudinal dimension of the movable portion being less than the longitudinal spacing between the first portion and second portion of the body portion, the movable portion being connected to the first portion and the second portion of the body portion and movable along the longitudinal axis of the body portion toward and away from the first portion and the second portion of the body portion, wherein a majority of the top surface and a majority of the bone-facing surface of the movable portion are exposed in the space between the first portion and the second portion of the body portion.

2. The bone plate system of claim 1 further comprising a drive mechanism engaging the movable portion for selectively moving the movable portion of the bone plate in two directions along the longitudinal axis of the bone plate.

3. The bone plate system of claim 2 wherein the drive mechanism comprises a threaded spindle engaging the movable portion of the bone plate so that rotational movement of the threaded spindle in one direction causes longitudinal movement of the movable portion of the bone plate in one direction for distraction of bone segments and rotational movement of the threaded spindle in the other direction causes longitudinal movement of the movable portion of the bone plate in another direction for compression of bone segments.

4. The bone plate system of claim 3 wherein the threaded spindle extends through a part of the body portion of the bone plate.

5. The bone plate system of claim 1 further comprising a rail connected to the body portion and extending across the enlarged through-opening in the body portion, the movable portion of the bone plate being mounted on the rail for movement along the longitudinal axis of the body portion.

6. The bone plate system of claim 1 wherein the bone screws have threaded shafts and non-threaded heads and the movable portion of the bone plate has an undercut defining a groove at the bone screw hole, the system further comprising a radially compressible split collet having an outer surface, an inner surface defining an axial opening and tabs extending radially outward from the outer surface, the tabs being sized and shaped to be receivable in the groove at the bone screw hole of the movable portion of the bone plate to lock the position of the collet with respect to the movable portion of the bone plate along an axis perpendicular to the longitudinal and transverse axes of the movable portion of the bone plate, the inner surface of the collet being sized and shaped to receive the head of one of the bone screws and to frictionally engage a portion of the bone screw, the outer surface of the collet being sized and shaped to be received within the bone screw hole and to engage the interior edge of the movable portion of the bone plate.

7. The bone plate system of claim 6 wherein the collet comprises an assembly of an annular inner member and an annular outer member, the annular outer member and the annular inner member having concentric portions, wherein the concentric portions of the collet assembly are sized and shaped so that the concentric portion of the annular outer member is radially compressible by the interior edge of the movable portion of the bone plate and so that the concentric portion of the annular inner member is radially compressible by radial compression of the annular outer member, and wherein the annular inner member and bone screw are sized and shaped so that radial compression of the annular inner member causes the annular inner member to engage the bone screw to limit movement of the bone screw with respect to the collet.

8. The bone plate system of claim 7 wherein the annular outer member comprises metal and the annular inner member comprises a non-metallic material.

9. The bone plate system of claim 8 wherein the annular outer member includes a plurality of spaced axial slots and the annular inner member includes a plurality of spaced axial slots.

10. The bone plate system of claim 1 wherein the body portion of the bone plate has a central portion positioned between the first portion and the second portion, the central portion being longitudinally spaced from both the first portion and the second portion, the longitudinal distance between the central portion and the first portion being greater than the longitudinal dimension of the bone screw hole and the longitudinal distance between the central portion and the second portion being greater than the longitudinal dimension of the bone screw hole, the bone plate further comprising a second movable portion in the second enlarged opening of the body portion, the second movable portion having an interior edge defining a bone screw hole, the second movable portion being mounted to the body portion and movable along the longitudinal axis of the body portion

11. The bone plate system of claim 10 wherein the body portion has a first end and a second end and includes a first end portion between the first end and the first enlarged opening and a second end portion between the second end and the second enlarged opening, wherein the first end portion includes an interior edge defining a bone screw hole and the second end portion includes an interior edge defining a second bone screw hole.

12. The bone plate system of claim 11 wherein the body portion of the bone plate includes spaced longitudinal sides along the first enlarged opening and along the second enlarged opening and wherein the first movable portion and the longitudinal sides include complementary structures to limit movement of the first movable portion to longitudinal movement and wherein the second movable portion and the longitudinal sides include complementary structures to limit movement of the second movable portion to longitudinal movement.

13. The bone plate system of claim 11 further comprising:
a first guide rod extending longitudinally outward from the first end portion and through a longitudinal bore in the first movable portion, the first movable portion being slidable on the first guide rod; and
a second guide rod extending longitudinally outward from the second end portion and through a longitudinal bore in the second movable portion, the second movable portion being slidable on the second guide rod.

14. A bone plate system comprising:
a plurality of bone screws having threaded shafts and non-threaded heads;
a bone plate having first and second ends, a longitudinal axis and a transverse axis, the bone plate comprising:
a first end portion having a bone screw hole;
a second end portion having a bone screw hole, the second end portion being spaced from the first end portion, the first and second end portions being aligned along the longitudinal axis of the bone plate;
a central portion spaced from the first end portion and the second end portion by longitudinal distances, the central portion being aligned with the first and second end portions along the longitudinal axis of the bone plate;
a first movable portion positioned between the first end portion and the central portion and longitudinally aligned with the first end portion and the central portion, the first movable portion having a top surface, a bottom surface, a longitudinal dimension, an interior edge defining a bone screw hole and an undercut defining a groove at the bone screw hole between the top and bottom surfaces;
the longitudinal dimension of the first movable portion being less than the longitudinal distance between the first end portion and the central portion of the bone plate;
the first movable portion being movable in a longitudinal direction toward the central portion and in a longitudinal direction away from the central portion;
a second movable portion positioned between the second end portion and the central portion and longitudinally aligned with the second end portion and the central portion, the second movable portion having a top surface, a bottom surface, a longitudinal dimension, an interior edge defining a bone screw hole and an undercut defining a groove at the bone screw hole between the top and bottom surfaces;
the longitudinal dimension of the second movable portion being less than the longitudinal distance between the second end portion and the central portion of the bone plate;
the second movable portion being movable in a longitudinal direction toward the central portion and in a longitudinal direction away from the central portion;
the system further comprising:
a first radially compressible split collet having an outer surface, an inner surface defining an axial opening and tabs extending radially outward from the outer surface, the tabs being sized and shaped to be receivable in the groove at the bone screw hole of the first movable portion of the bone plate to lock the position of the first collet with respect to the first movable portion of the bone plate along an axis perpendicular to the longitudinal and transverse axes of the bone plate, the inner surface of the first collet being sized and shaped to receive the head of one of the bone screws and to frictionally engage a portion of the bone screw when radially compressed, the outer surface of the first collet being sized and shaped to be received within the bone screw hole of the first movable portion of the bone plate and to engage the first movable portion of the bone plate at the bone screw hole; and
a second radially compressible split collet having an outer surface, an inner surface defining an axial opening and tabs extending radially outward from the outer surface, the tabs being sized and shaped to be receivable in the groove at the bone screw hole of the second movable portion of the bone plate to lock the position of the second collet with respect to the second movable portion of the bone plate along an axis perpendicular to the longitudinal and transverse axes of the bone plate, the inner surface of the second collet being sized and shaped to receive the head of one of the bone screws and to frictionally engage a portion of the bone screw when radially compressed, the outer surface of the second collet being sized and shaped to be received within the bone screw hole of the second movable portion of the bone plate and to engage the second movable portion of the bone plate at the bone screw hole.

15. The bone plate system of claim 14 wherein:
the first collet comprises an assembly of an annular inner member and an annular outer member, the annular outer member and the annular inner member having concentric portions, wherein the concentric portions of the first collet assembly are sized and shaped so that the concentric portion of the annular outer member is radially compressible by the interior edge of the first movable portion of the bone plate and so that the concentric portion of the annular inner member is radially compressible by radial compression of the annular outer member, and wherein the annular inner member and bone screw are sized and shaped so that radial compression of the annular inner member causes the annular inner member to engage the bone screw to limit movement of the bone screw with respect to the first collet; and
the second collet comprises an assembly of an annular inner member and an annular outer member, the annular outer member and the annular inner member having concentric portions, wherein the concentric portions of the second collet assembly are sized and shaped so that the concentric portion of the annular outer member is radially compressible by the interior edge of the second movable portion of the bone plate and so that the concentric portion of the annular inner member is radially compressible by radial compression of the annular outer member, and wherein the annular inner member and bone screw are sized and shaped so that radial compression of the annular inner member causes the annular inner member to engage the bone screw to limit movement of the bone screw with respect to the second collet.

16. The bone plate system of claim 15 wherein the annular outer member of each collet assembly comprises metal and the annular inner member of each collet assembly comprises a non-metallic material.

17. The bone plate system of claim 16 wherein the annular outer member of each collet assembly includes a plurality of spaced axial slots and the annular inner member of each collet assembly includes a plurality of spaced axial slots.

18. The bone plate system of claim 16 wherein:
each collet assembly has a first end at the annular outer member, a second end at the annular inner member and an axial dimension between the first end and the second end;
the distance between the top surface and the bottom surface of the first movable portion of the bone plate is at least equal to the axial dimension of at least one of the collet assemblies; and
the distance between the top surface and the bottom surface of the second movable portion of the bone plate is at least equal to the axial dimension of at least one of the collet assemblies.

19. The bone plate system of claim 14 wherein the first movable portion has a threaded longitudinal bore between the top and bottom surfaces and the second movable portion has a threaded longitudinal bore between the top and bottom surfaces, the system further comprising:
a first spindle drive mechanism having threads engaging the threaded bore of the first movable portion for selectively moving the first movable portion, wherein rotational movement of the first spindle drive mechanism in one direction causes longitudinal movement of the first movable portion of the bone plate away from the central portion for distraction of bone segments and rotational movement of the first spindle drive mechanism in the other direction causes longitudinal movement of the first movable portion of the bone plate toward the central portion for compression of bone segments; and
a second spindle drive mechanism having threads engaging the threaded bore of the second movable portion for selectively moving the second movable portion wherein rotational movement of the second spindle drive mechanism in one direction causes longitudinal movement of the second movable portion of the bone plate away from the central portion for distraction of bone segments and rotational movement of the second spindle drive mechanism in the other direction causes longitudinal movement of the second movable portion of the bone plate toward the central portion for compression of bone segments;

20. The bone plate system of claim 14 wherein:
the bone plate includes spaced longitudinal sides between the first end portion and the central portion, and wherein the first movable portion and the longitudinal sides include complementary structures to limit movement of the first movable portion to longitudinal movement; and
the bone plate includes spaced longitudinal sides between the second end portion and the central portion, and wherein the second movable portion and the longitudinal sides include complementary structures to limit movement of the second movable portion to longitudinal movement.

21. The bone plate system of claim 14 wherein the bone plate includes:
a first guide rail extending longitudinally outward from the first end portion and through a longitudinal bore in the first movable portion, the first movable portion being slidable on the first guide rod; and
a second guide rail extending longitudinally outward from the second end portion and through a longitudinal bore in the second movable portion, the second movable portion being slidable on the second guide rod.

22. A bone plate system comprising:
a plurality of bone screws having threaded shafts and non-threaded heads;
a bone plate having first and second ends, a longitudinal axis, a transverse axis, a top surface, a bottom surface, an interior surface defining a bone screw hole extending from the top surface to the bottom surface, and an undercut defining a groove at the bone screw hole between the top and bottom surfaces;
a radially compressible collet assembly including an annular metal outer member and an annular non-metallic inner member, the outer member having an outer surface and tabs extending radially outward from the outer surface, the tabs being sized and shaped to be receivable in the groove at the bone screw hole of the bone plate to lock the position of the collet assembly with respect to the bone plate along an axis perpendicular to the longitudinal and transverse axes of the bone plate, the inner member having an inner surface defining an axial opening, the inner surface of the inner member being sized and shaped to receive the head of one of the bone screws and to frictionally engage a portion of the bone screw when radially compressed, the outer surface of the outer member being sized and shaped to be received within the bone screw hole of the bone plate and to engage the interior surface of the bone plate;
the inner member and the outer member having spaced axial slots and concentric portions, the concentric portions being sized and shaped so that the concentric portion of the outer member is radially compressible by the interior surface of the bone plate and so that the concentric portion of the inner member is radially compressible by radial compression of the outer member, and wherein the inner member and bone screw are sized and shaped so that radial compression of the inner member causes the inner member to engage the bone screw to limit movement of the bone screw with respect to the collet assembly;
the collet assembly having a first end at the annular outer member, a second end at the annular inner member and an axial dimension between the first end and the second end; and
the distance between the top surface and the bottom surface of the bone plate being at least equal to the axial dimension of the collet assembly.

23. The bone plate system of claim 22 wherein the bone plate comprises:
a body portion having an enlarged through-opening, the enlarged through-opening having longitudinal and transverse dimensions larger than the longitudinal and transverse dimensions of the bone screw hole; and
a movable portion in the enlarged through-opening of the body portion, the movable portion having a top surface, a bone-facing surface, a longitudinal dimension, a transverse dimension and an interior edge defining a bone screw hole extending from the top surface to the bone-facing surface, the longitudinal dimension of the movable portion being less than the longitudinal dimension of the enlarged through-opening in the body portion, the movable portion being movable in the enlarged through-opening along the longitudinal axis of the body portion, wherein a majority of the top surface and a majority of the bone-facing surface of the movable portion are exposed in the enlarged through-opening.

24. The bone plate system of claim 22 wherein the bone plate comprises:
a first end portion having a bone screw hole;
a second end portion having a bone screw hole, the second end portion being spaced from the first end portion, the first and second end portions being aligned along the longitudinal axis of the bone plate;
a central portion spaced from the first end portion and the second end portion by longitudinal distances, the central portion being aligned with the first and second end portions along the longitudinal axis of the bone plate;
a first movable portion positioned between the first end portion and the central portion and longitudinally aligned with the first end portion and the central portion, the interior surface and the first bone screw hole being associated with the first movable portion;
the longitudinal dimension of the first movable portion being less than the longitudinal distance between the first end portion and the central portion of the bone plate;
the first movable portion being movable in a longitudinal direction toward the central portion and in a longitudinal direction away from the central portion; and
a second movable portion positioned between the second end portion and the central portion and longitudinally aligned with the second end portion and the central portion, the second movable portion having an interior surface defining a bone screw hole and an undercut defining a groove at the bone screw hole;
the longitudinal dimension of the second movable portion being less than the longitudinal distance between the second end portion and the central portion of the bone plate;
the second movable portion being movable in a longitudinal direction toward the central portion and in a longitudinal direction away from the central portion.

25. The bone plate system of claim 24 wherein the first movable portion has a longitudinal bore, the second movable portion has a longitudinal bore, and wherein the plate includes:
a first longitudinal member extending from the first end portion, through the longitudinal bore of the first movable portion and to the central portion; and
a second longitudinal member extending from the second end portion, through the longitudinal bore of the second movable portion and to the central portion.

26. The bone plate system of claim 25 wherein the longitudinal bores are threaded and wherein the first and second longitudinal members include threaded portions.
